(19) United States
(12) Patent Application Publication
(10) Pub. No.: US 2014/0364844 A1
(43) Pub. Date: Dec. 11, 2014

(54) MINIATURIZED DUAL-MODE ELECTROSURGICAL DEVICE AND METHODS OF USING SAME

(71) Applicant: ElectroMedical Associates LLC, (unknown)

(72) Inventor: Robert A. VAN WYK, St. Pete Beach, FL (US)

(73) Assignee: ElectroMedical Associates LLC, Bethesda, MD (US)

(21) Appl. No.: 14/297,785

(22) Filed: Jun. 6, 2014

Related U.S. Application Data
(60) Provisional application No. 61/956,357, filed on Jun. 6, 2013.
(60) Provisional application No. 61/956,358, filed on Jun. 6, 2013.

(51) Int. Cl. A61B 18/14; A61B 18/12

(52) U.S. Cl. CPC A61B 18/1477; A61B 18/1206; A61B 2018/00577
USPC 606/37; 606/40

(57) ABSTRACT

Described herein is a miniaturized dual-mode high-efficiency electrosurgical device that uses radio frequency (RF) energy to cut, resect, ablate, vaporize, denaturize, drill, coagulate and/or form lesions in soft tissues by means of a current-dispersing active element, with or without the use of externally supplied liquids, when operating in a first mode and then uses energy and same active element to thermally cauterize and/or spot coagulate tissues as needed when operating in a second mode. The miniaturized dual-mode RF electrosurgical devices of the instant invention find particular utility in the field neurosurgery, more particularly in the removal of brain tumors.

### PRIORITY

This application claims the benefit of U.S. Provisional Application Ser. Nos. 61/956,357 and 61/956,358, both filed Jun. 6, 2013, the entire contents of which are incorporated by reference herein.

### FIELD OF THE INVENTION

The present invention relates generally to the field of electrosurgery with reference to instruments and systems for energy-based medical therapy, and is more particularly directed to miniaturized high efficiency electrosurgical devices and methods that use radio frequency (RF) energy to cut, resect, ablate, vaporize, denaturize, drill, coagulate and/or form lesions in soft tissues, with or without the use of externally supplied liquids when operating in a first mode and then thermally cauterize and/or spot coagulate tissues as needed when operating in a second mode. The miniaturized dual-mode RF electrosurgical devices of the instant invention find particular utility in neurosurgery procedures, more particularly the removal of brain tumors.

### BACKGROUND OF THE INVENTION

Certain neurological procedures require the removal of a tissue mass from a highly vascular region. One such procedure is the removal of a brain tumor. Under current practices, a first device, generally a bipolar coagulation device, is used to coagulate tissue prior to resection with a second device, generally a conventional cutting device. This approach is not optimal since bleeding generally commences with each incision made and frequently recurs with each device that is removed, adjusted and/or reintroduced. As such, the introduction, removal and/or exchange of multiple instruments is not ideal. Furthermore, while monopolar electrosurgical cutting devices are the gold standard for many procedures, as discussed hereinbelow, they are not suited to surgery in the brain.

Monopolar electrosurgical devices, such as Bovie knives, needles and loop electrodes are preferred for cutting since they afford a simultaneous coagulating effect when tissue is cut electrosurgically. However, such monopolar devices require the use of a remote return electrode (e.g., a dispersive pad), wherein current flows from the device through adjoining tissue on its way to the return electrode. While current diffusion is generally not a concern when applied to muscular, connective, or epithelial tissues, such current flow through nervous tissues, particularly the intricate tissues of the brain, can be very hazardous indeed. The brain is a complex mass of diverse tissues, one that is both non-uniform in conductivity and non-homogeneous in structure. Owing to this variation, stray currents arising from electrosurgery will not disperse uniformly as they do in other parts of the body; in fact, such current can concentrate in certain structures thereby causing what is often uncontrolled or irreparable damage to the delicate nervous tissue.

Given the high risk of complication, the use of monopolar electrosurgical devices is not an option for neurosurgery. However, the use of multiple devices is also not preferred. Despites advances in technology, satisfactory single device alternatives that allow for concomitant tissue resection and spot coagulation and are also sized and safe for use in the brain continue to be absent from the art.

### SUMMARY OF THE INVENTION

The present invention is directed to a miniaturized electrosurgical device that finds particular utility in the neurological arts—one that is capable of concurrently or concomitantly cutting tissue and achieving haemostasis, or alternatively, performing highly accurate vaporization of tissue with concurrent haemostasis, all at very low power levels, typically less than 10 Watts. As noted above, stray currents arising from electrosurgery in the brain can cause damage to surrounding tissue; accordingly, it is highly desirable to use very low power levels and to confine the current flow (to the maximum extent possible) to the region surrounding the active electrode. It is also preferable to utilize a bipolar construction, particularly one in which the return electrode on the electrosurgical device in positioned in close proximity to the active electrode so as to confine the electrical field generated and concentrate the current flow to the region between the active and return electrodes.

Due to the desiccating effect of the current flowing through the immediately surrounding tissue, this close proximity and the resulting locally relatively high current densities can result in some coagulation. However, because the power level is extremely low, the haemostatic effect is limited. Also, spot coagulation of bleeding regions is frequently necessary after a cut is made and, as noted above, accomplishing this through the application of a monopolar coagulation device to the region is undesirable because of the current flow into the surrounding tissue on its way to the remotely located return pad.

Accordingly, it is an objective of the present invention to provide a dual-mode electrosurgical device configured and suitable for use in neurosurgical applications that has two selectable modes of operation: an electrosurgical mode for cutting and coagulating tissue, and a thermal mode for coagulation of tissue using thermal energy. According to the principles of the present invention, the distal element functions as an active current-dispersing electrode when working in the electrosurgical mode and as an active element that is itself heated when working in the thermal (electrocautery) mode.

The present invention contemplates methods of using such devices, particularly bipolar embodiments of such devices, in both wet and semi-dry environments, with particular application to brain surgery. The present invention further contemplates monopolar embodiments of the dual-mode device that operate in conjunction with a remotely located return pad for use in dry environments, particularly superficial tissues that are the target of dermatological and dental electrosurgery. In such “dry” applications, the absence of conductive fluid mitigates the problem of unwanted current diffusion.

These and other goals and objectives may be accomplished by the invention herein disclosed. However, it will be understood by those skilled in the art that one or more aspects of this invention can meet certain of the above objectives, while one or more other aspects can meet certain other objectives. Each objective may not apply equally, in all its respects, to every aspect of this invention. As such, the preceding objectives and foregoing description should be viewed in the alternative with respect to any one aspect of this invention.

The foregoing objects, aspects and features of the invention discussed herein above will become more fully apparent when the following detailed description is read in conjunction with the accompanying figures and/or examples. However, it is to be understood that both the foregoing summary of the invention and the following detailed description are of preferred embodiments and not restrictive of the invention or other alternate embodiments of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of electrode design. Such objects, features, benefits and advantages apparent from the above in conjunction with the accompanying examples, data, figures and all reasonable inferences to be drawn there-from are specifically incorporated herein.

### BRIEF DESCRIPTION OF THE DRAWINGS

Various aspects and applications of the present invention will become apparent to the skilled artisan upon consideration of the brief description of the figures and the detailed description of the present invention and its preferred embodiments that follows:

FIG. 1 depicts in perspective view a miniaturized electrosurgical device constructed in accordance with the principles of this invention.

FIG. 2 is a plan view of the miniaturized electrosurgical device of FIG. 1.

FIG. 3 is a side elevational view of the miniaturized electrosurgical device of FIG. 1.

FIG. 4 is a distal axial view of the miniaturized electrosurgical device of FIG. 1.

FIG. 5 is a proximal axial view of the miniaturized electrosurgical device of FIG. 1.

FIG. 6 is a plan view of a distal portion of the miniaturized electrosurgical device of FIG. 1.

FIG. 7 is a side elevational sectional view of the distal portion of the objects of FIG. 6 at location A-A of FIG. 6.

FIG. 8 is a perspective view of the distal portion of the objects of FIG. 6.

FIG. 9 is a plan view of the objects of FIG. 8.

FIG. 10 is a side elevational view of the objects of FIG. 8.

FIG. 11 is an expanded view of the sectional view of FIG. 7.

FIG. 12 is a side elevational sectional view of the distal portion of the miniaturized electrosurgical device of FIG. 1 during use showing irrigant flow.

FIG. 13 is a side elevational sectional view of the distal portion of the miniaturized electrosurgical device of FIG. 1 during use showing electrical current flow.

FIG. 14 is a perspective view of the distal portion of an alternate embodiment of a miniaturized electrosurgical device constructed in accordance with the principles of this invention.

FIG. 15 is a plan view of the objects of FIG. 14.

FIG. 16 is a side elevational view of the objects of FIG. 14.

FIG. 17 is a side elevational sectional view of the objects of FIG. 14 at location A-A of FIG. 15.

FIG. 18 is a perspective view of an alternate embodiment of a miniaturized electrosurgical device formed in accordance with the principles of this invention.

FIG. 19 is an expanded perspective view of the distal portion of the objects of FIG. 18 at location B of FIG. 18.

FIG. 20 is a plan view of the objects of FIG. 18.

FIG. 21 is a side elevational sectional view of the objects of FIG. 18 at location B-B of FIG. 20.

FIG. 22 is an expanded view of the distal portion of FIG. 21 at location A of FIG. 21.

FIG. 23 is a plan view of the objects of FIG. 18.

FIG. 24 is a side elevational sectional view of the objects of FIG. 18 at location C-C of FIG. 23.

FIG. 25 is a plan view of the objects of FIG. 18.

FIG. 26 is a diagrammatic view of the handle portion of the alternate miniaturized electrosurgical device of FIG. 18, with reference to its internal circuitry.

FIG. 27 is a diagrammatic view of the internal circuitry of the handle portion of the alternate miniaturized electrosurgical device of FIG. 18 showing current flow paths when operating in a first electrosurgical mode cutting tissue.

FIG. 28 depicts the distal portion of the alternate miniaturized electrosurgical device of FIG. 18 showing current flow paths when operating in a first electrosurgical mode cutting tissue.

FIG. 29 is a diagrammatic view of the internal circuitry of the handle portion of the alternate miniaturized electrosurgical device of FIG. 18 showing current flow when the device is operated in a second thermal mode.

FIG. 30 depicts the distal portion of the alternate miniaturized electrosurgical device of FIG. 18 showing the electrical current flow path and thermal energy flow when operating in a second thermal mode treating tissue.

FIG. 31 is a diagrammatic view of a second alternate embodiment for the internal circuitry of a handle portion suitable for use with the alternate miniaturized electrosurgical device of FIG. 18.

FIG. 32 is a diagrammatic view of the handle portion of FIG. 31 showing current flow when the device is operated in a first electrosurgical mode.

FIG. 33 depicts the distal portion of an alternate miniaturized electrosurgical device that includes the alternate handle portion embodiment of FIG. 31 (not shown), showing current flow paths when operating in a first electrosurgical mode cutting tissue

### DETAILED DESCRIPTION OF THE PREFERRED EMBODIMENTS

The present invention is directed to a dual-mode electrosurgical device capable of operating in both an electrical cutting mode and a thermal cautery mode. Though bipolar embodiments are preferred, monopolar embodiments are likewise contemplated.

In the context of the present invention, the term “electrosurgery” refers to the application of a high-frequency electric current, particularly a radio-frequency (RF) current, to biological tissue as a means to cut, coagulate, desiccate, or fulgurate tissue. Its benefits include the ability to make precise cuts with limited blood loss. Although the tissue is heated by an electric current in the context of electrosurgical procedures, and thus such electrical devices can, in limited instances, be used for the cauterization of tissue, such cauterization (i.e., desiccation) may be difficult to control or direct to particular tissues; as such, it cannot serve the need for spot coagulation that often arises in the course of an invasive or semi-invasive surgical procedure. Moreover, herein and elsewhere in the art, “electrosurgery” and “electrocautery” are considered to be two very distinct processes. Whereas “electrocautery” instruments and procedures use heat conduction from a probe heated to an elevated temperature by a direct current (much in the manner of a soldering iron), electrosurgical instruments and procedures, by contrast, use alternating current to generate an electric field that directly heats the tissue itself. In those instances wherein the use of electrosurgical energy results in destruction of small blood vessels and halting of bleeding, the process is referred to herein as “electrocoagulation”, a by-product or side-effect of electrosurgery that is distinguished in the art from true “electrocautery”.

Electrosurgery is commonly used in dermatological, gynecological, cardiac, plastic, ocular, spine, ENT, maxillofacial, orthopedic, urological, neuro- and general surgical procedures as well as certain dental procedures. Accordingly, the devices and methods of the present invention are widely applicable to a number of interventions. However, preferred embodiments of the present invention find particular utility in connection with neurosurgery, particularly the destruction and/or removal of brain tumors.

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### Elements of the Present Invention:

Unless otherwise defined, all technical and scientific terms used herein have the same meaning as commonly understood by one of ordinary skill in the art to which this invention belongs. In case of conflict, the present specification, including following definitions, will control.

The words “a”, “an”, and “the” as used herein mean “at least one” unless otherwise specifically indicated.

The term “proximal” refers to that end or portion which is situated closest to the user. For example, the proximal end of an instrument designed in accordance with the present invention would typically include the power module and handle.

The term “distal” refers to that end or portion situated farthest away from the user. For example, the distal end of an instrument designed in accordance with the present invention would typically include the “active” element(s) of the instrument.

The present invention in certain embodiments may make reference to a “structural member”, “elongate portion” or “shaft” that directly conducts energy to the active electrode. The structural member is elongate, of a linear or angled, and rounded, rod-like or tubular construction. The elongate shaft is preferably conductive and more preferably formed of metal or metallic material. In certain embodiments, the shaft may be hollow, including a lumen running therethrough that serves as a channel for the inner element or an aspiration path for removing gaseous and liquid ablation byproducts. The latter lumen flow may also serve to cool the device. However, non-lumened and non-aspirating inner element embodiments are also contemplated. The shaft which conducts power may be surrounded by and electrically isolated from a coaxially positioned an external metallic tubular element which may in certain embodiments be part of the return current path to the generator, a distal portion of the external metallic tubular element serving as a return electrode.

In common terminology and as used herein, the term “electrode” may refer to one or more components of an electrosurgical instrument (such as an active element or a return electrode) or to the entire device, as in an “ablator electrode” or “cutting electrode”. Such electrosurgical instruments are often interchangeably referred to herein as “probes”, “devices” or “instruments”.

The present invention makes reference to one or more “active elements”. As used herein, the term “active element” refers to one or more conductive elements formed from any suitable preferably spark-resistant metal material, such as stainless steel, nickel, titanium, molybdenum, tungsten, and the like as well as combinations thereof. In the context of the present invention, the “active element” is generally disposed at the distal end of the instrument and comprises a current-dispersing electrode when the instrument is operating in the electrosurgical mode and as a heated cautery element when the instrument is operating in the thermal mode. In the context of the present invention, the distal active element is connected, for example via wiring disposed within the control/handle portion of the instrument, to a power supply, for example, an externally located electrosurgical generator or a rechargeable battery disposed within the power module component, either singly or in combination capable of generating the requisite electric field or thermal energy, depending on the mode employed.

Like the overall electrosurgical instrument, the size, shape and orientation of the active electrode itself and the active surface (i.e., cutting or cauterizing surface) defined thereby may routinely vary in accordance with the need in the art. It will be understood that certain geometries may be better suited to certain utilities. Accordingly, those skilled in the art may routinely select one shape over another in order to optimize performance for specific surgical procedures.

The present invention contemplates electrosurgical instruments of the “bipolar’ and “monopolar” configuration. Both configurations require the use of a “return electrode”, i.e., one or more powered conductive elements to which current flows after passing from the active electrode(s) back to the general-purpose generator. In the context of a monopolar device, the return electrode takes the form of a patient-mounted return pad. In the context of a bipolar device, the return electrode is located on the electrosurgical instrument itself and is preferably formed from a suitable electrically conductive material, for example a metal material such as stainless steel, nickel, titanium, molybdenum, tungsten, aluminum and the like as well as combinations thereof.

Electrosurgical instruments contemplated by the present invention may be fabricated in a variety of sizes and shapes to optimize performance in a particular surgical procedure. For instance, instruments configured for use in small vascular spaces such as the brain may be highly miniaturized while those adapted for shoulder, knee and other large joint use may need to be larger to allow high rates of tissue removable. Likewise, electrosurgical instruments for use in arthroscopy, otolaryngology and similar fields may be produced with a rounded geometry, e.g., circular, cylindrical, elliptical and/or spherical, using turning and machining processes, while such geometries may not be suitable for other applications. Accordingly, the geometry (i.e., profile, perimeter, surface, area, etc.) may be square, rectangular, polygonal or have an irregular shape to suit a specific need.

In certain embodiments, the present invention makes reference to one or more “floating electrodes”. As used herein, the term “floating electrode” refers to one or more disconnected (unpowered) electrodes that may contact the surrounding conducting liquid and/or tissue. The electrical potential of such disconnected electrodes is “floating” and is determined by the size and position of the electrode, the tissue type and properties, and the presence or absence of bodily fluids or externally supplied fluid. “Floating” electrodes for electrosurgery are described in published U.S. Pat. Nos. 7,563,261 and 8,308,724, the contents of which are incorporated by reference herein in their entirety. In the context of the present invention, the “floating” electrode is preferably mounted in such a way that one portion of the electrode is in close proximity to the tip of the active electrode, in the region of high potential. Another portion of the floating electrode is preferably placed farther away, in a region of otherwise low potential. This region of low potential may be in contact with the fluid environment, in contact with tissue, or both.

In the context of the present invention, a floating electrode can generate and concentrate high power density in the vicinity of the active region, and results in more efficient liquid heating, steam bubble formation and bubble trapping in this region. This increases the probe efficiency, which, in turn, allows the surgeon to substantially decrease the applied RF power and thereby reduce the likelihood of patient burns and unintended local tissue injury. The probe may be operated so that the portion of the floating electrode in close proximity to the active electrode has sufficient current density to produce vaporization of the liquid and arcing so as to vaporize tissue. Alternatively, the probe may be operated so that the floating electrode contacts tissue, wherein those portions of the floating electrode in contact with the tissue have sufficient current density to desiccate blood vessels and tissue so as to achieve coagulation. This is particularly useful for achieving haemostasis in vascular tissue, such as, for instance, that present when performing tonsillectomies. However, in that the floating electrode is not itself powered but rather is only indirectly heated, it cannot achieve and/or maintain the elevated temperatures required for cauterization.

In certain embodiments, the present invention makes reference to one or more “insulators” separating active and floating, or active and return electrodes. As used herein, the term “insulator” refers to a non-conductive element formed from a suitable dielectric material, examples of which include, but are not limited to, alumina, zirconia, and high-temperature polymers.

In certain embodiments, the present invention makes reference to “fluid(s)”, particularly in connection with the “wet environment” embodiments. As used herein, the term “fluid” encompasses liquids, gases and combinations thereof, either electrically conductive or non-conductive, intrinsic to the tissue or externally supplied. In the context of the present invention, the term “fluid” extends to externally supplied liquids such as saline as well as body fluids, examples of which include, but not limited to, blood, peritoneal fluid, lymph fluid, pleural fluid, gastric fluid, bile, and urine.

The present invention makes reference to the ablation, coagulation, vaporization and cauterization of tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application, including the resection and/or vaporization any target tissue with particular applicability to the ablation, vaporization, destruction and removal of brain tumors.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

### Utilities of the Present Invention:

Devices of the instant invention have the ability to cut, vaporize or thermally affect tissue while minimizing harm to adjacent tissues. For cutting and vaporizing extremely low power levels are used, the devices being extremely efficient in their use of RF energy. Other less efficient electrosurgical devices use higher power levels, the excess power beyond that required for cutting or vaporization being deposited in adjacent tissues where it may cause unintended desiccation, thermal damage and necrosis. Devices of the instant invention use small active electrodes with geometries that produce high current densities that, in turn, result in highly efficient cutting or vaporization.

As noted above, the electric field surrounding the active electrode may be further intensified by the use of a floating (or focusing) electrode in close proximity to the active electrode, and/or by incorporating a return electrode on the device in proximity to the active or floating electrode. When a return electrode is positioned on the device in close proximity to the active or floating electrode, the electric field is intensified in the region between the electrodes so as to minimize current flow through adjacent tissues. This intensified current flow in the region between the electrodes may have sufficient intensity to create a haemostatic effect on tissue simultaneously with the tissue cutting due to desiccation of adjacent tissue.

While this simultaneous haemostatic effect may be sufficient to control bleeding in may cases, in many other situations, there is a need to locally treat (coagulate) tissue to prevent or halt bleeding. While this may frequently be accomplished using a second device such as a bipolar forceps or a monopolar cautery, the need to use a second device creates delay and increases procedure time. Also, in the case of the monopolar cautery, because the current is flowing through the patient to a remotely located return pad, there is opportunity for unintended tissue damage in regions remote to the treatment site. This is particularly true in the brain where the structure is non-homogeneous and current may follow electrically preferred but clinically undesirable paths.

Central to the present invention is the discovery that it is possible to produce an electrosurgical device that operates in two modes so as to allow highly efficient electrosurgical cutting and vaporization of tissue, and also the thermal coagulation of tissue. Because the coagulation of tissue is accomplished through the application of thermal rather than RF energy and no electrical current flows to the patient, the possibility of unintended tissue damage due to current flow through adjacent tissue is eliminated. In a first embodiment, for use in wet or semi-dry environments a device with the return electrode on the device in proximity to the active electrode is required. In a second embodiment for use in dry fields, the return to the electrosurgical generator is through a remotely located return pad.

The present invention is not restricted to one particular field of surgery but rather finds utility in connection with a wide variety of applications, from oncological to reconstructive, cosmetic, arthroscopic, ENT, urological, gynecological, and/or laparascopic procedures, as well as in the context of general open surgery. However, certain preferred embodiments of the present invention are particularly configured for neurosurgery, particularly brain tumor removal, wherein the devices are preferably miniaturized in scale and bipolar in construction, with an active element, return electrode and optional floating electrode in close proximity so as to tightly restrict the applied electrical field to the target tissue of interest and thereby avoid “stray” or other unwanted current flow.

Electrosurgical instruments designed in accordance with the principles of the present invention_find utility in connection with a variety of medical, both human and veterinary, applications for cutting, cauterization, coagulation, evaporation, sculpting, shrinking, smoothing, lesion formation, among others, in various types of tissue. The instruments can be used in a variety of medical procedures, like minimally invasive or open surgery, cosmetic, dental or dermatological, on the surface or inside the body. Though the present invention is not particularly limited to the treatment of any one specific disease, body part or organ or the removal of any one specific type of tissue, certain preferred embodiments in the present invention are particularly configured for brain surgery and particularly the removal of brain tumors.

The active area of the instrument (i.e., the active element at the distal end) can take many shapes and forms, and can be configured to meet the needs of the specific procedure in such fields. Thus, for the most part, choices in geometry constitute a design preference. However, in the context of the present invention and particularly the neurosurgical applications, it is desirable to utilize a miniaturized bipolar electrode assembly such as discussed above.

Electrosurgical instruments formed in accordance with the principles of this invention are generally comprised of a proximal handle portion and an elongate distal portion designed to be inserted into the environment of interest. The proximal end of the instrument is typically connected to an electrosurgical generator, wherein the handle portion is provided with one or more buttons (or switches or other activating elements) on its surface that control the output of the electrosurgical generator. Alternatively, the electrosurgical generator may be controlled by a foot-activated control. In either case, depending on the environment, the desires of the surgeon, and the condition being treated, instruments of the present invention can be operated continuously or intermittently, at variable powers and intensities.

In accordance with the objectives of the present invention, the electrosurgical generator is preferably configured to deliver radio-frequency energy and preferably operates at very low power levels, for example, less than 15 Watts, preferably less than 10 Watts, more preferably between 1 and 8 Watts.

While some embodiments of the present invention are designed to operate in dry or semi-dry environments, other bipolar embodiments utilize the endogenous fluid and/or an exogenous irrigant of a “wet field” environment to transmit current to the return electrode and therethrough to the RF energy source. In certain embodiments, the “irrigant” (whether native or externally applied) is heated to the boiling point, whereby thermal tissue treatment arises through direct contact with either the boiling liquid itself or steam associated therewith. This thermal treatment may include desiccation to stop bleeding (haemostasis), and/or shrinking, denaturing, or enclosing of tissues for the purpose of volumetric reduction (as in the soft palate to reduce snoring) or to prevent aberrant growth of tissue, for instance, malignant tumors.

Liquids (either electrically conductive or non-conductive) and gaseous irrigants, either singly or in combination may also be advantageously applied to instruments for incremental vaporization of tissue. Normal saline solution may be used. Alternatively, the use of low-conductivity irrigants such as water or gaseous irrigants or a combination of the two allows increased control of the electrosurgical environment.

The electrosurgical instruments of the present invention may be used in conjunction with existing diagnostic and imaging technologies, for example imaging systems including, but not limited to, MRI, CT, PET, x-ray, fluoroscopic, thermographic, photo-acoustic, ultrasonic and gamma camera and ultrasound systems. Such imaging technology may be used to monitor the introduction and operation of the instruments of the present invention. For example, existing imaging systems may be used to determine location of target tissue, to confirm accuracy of instrument positioning, to assess the degree of tissue vaporization (e.g., sufficiency of tissue removal), to determine if subsequent procedures are required (e.g., thermal treatment such as coagulation and/or cauterization of tissue adjacent to the target tissue and/or surgical site), and to assist in the atraumatic removal of the instrument.

### Illustrative Embodiments of the Present Invention

Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

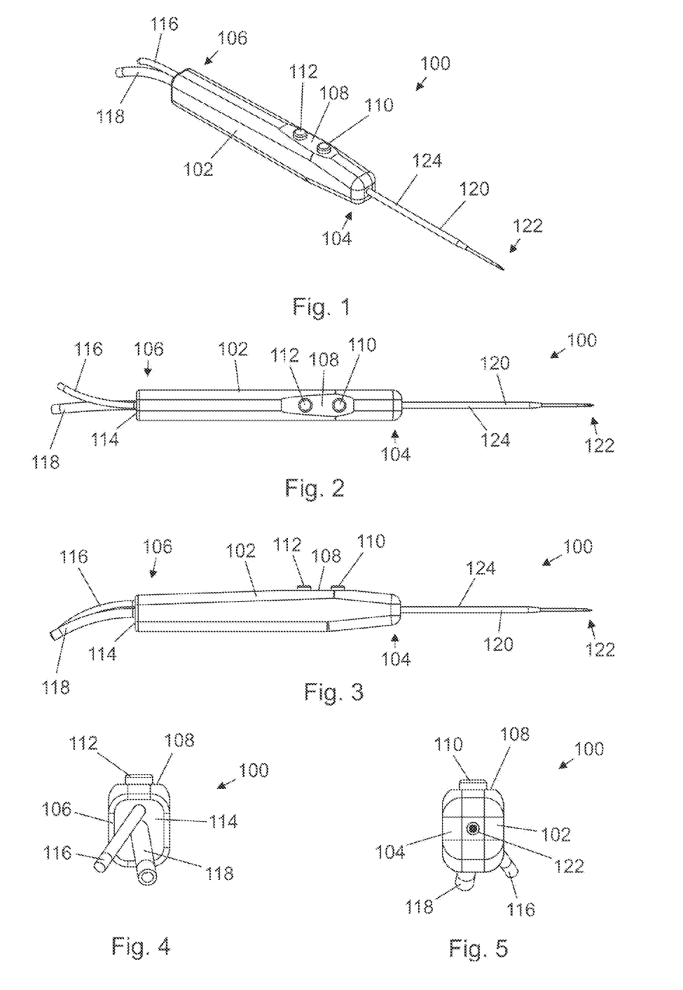

FIGS. 1 through 5 depict a bipolar electrosurgical device 100 constructed in accordance with the principles of this invention. Device 100 has a proximal portion 102 forming a handle having a distal end 104, a proximal end 106, and a top surface 108 from which protrude first activation button 110 and second activation button 112. Proximal end 106 has a proximal-most surface 114 from which pass cable 116 and tubular member 118. Activation buttons 110 and 112 are electrically connected via cable 116 to an electrosurgical generator (not shown) such that depressing first button 110 causes the generator to supply a first radio frequency (RF) waveform having a first preset energy level to device 100, and depressing second button 112 causes the generator to supply a second RF waveform having a second preset energy level to device 100. Distal portion 120 has a distal end 122 having mounted thereto an electrode assembly.

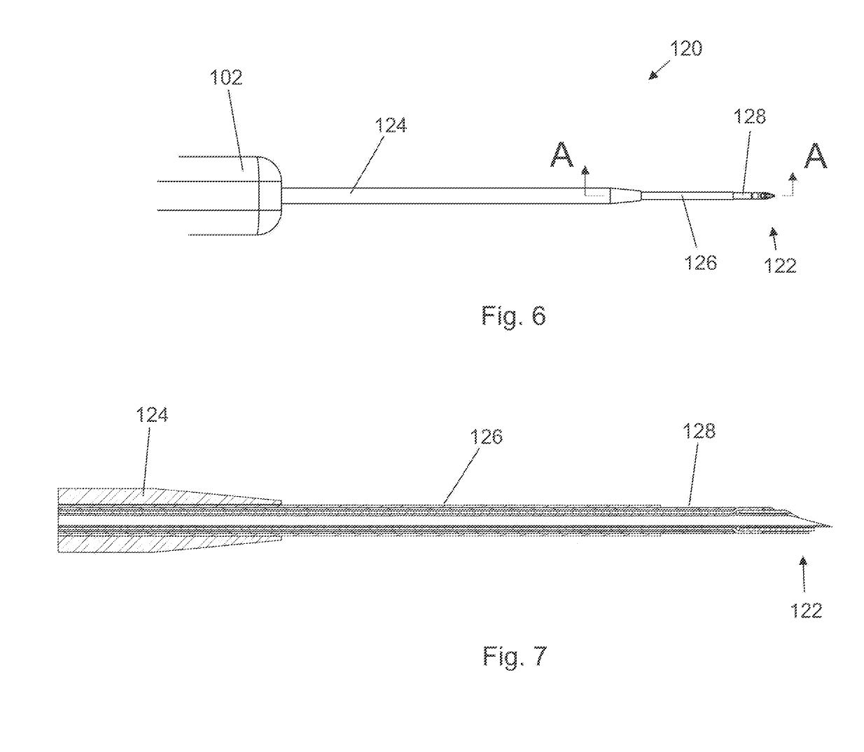

FIGS. 6 and 7 depict distal portion 120 of device 100 and the construction thereof. Distal portion 120 is an assembly of elongate tubular elements electrically isolated from each other and extending from proximal handle portion 102 at their proximal end. Outer tubular element 124 is electrically isolated from tubular member 128 by dielectric layer 126, element 124 serving to provide rigidity to distal portion 120.

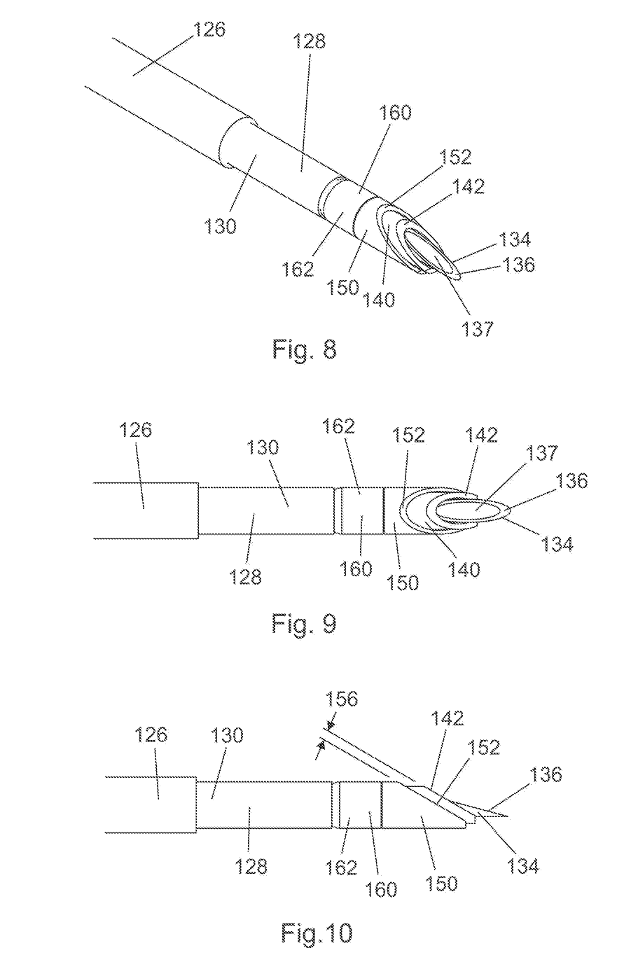

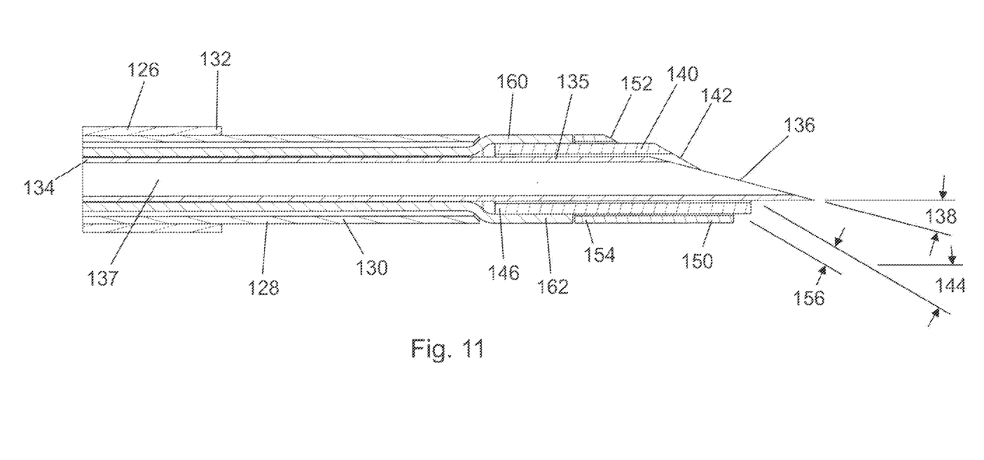

FIGS. 8 through 11 depict the distal portion 122 of distal portion 120. Tubular metallic element 134 with lumen 137 has a proximal end connected by circuitry within proximal handle portion 102 and cable 116 to an electrosurgical generator (not shown) such that when first button 110 or second button 112 is depressed, RF energy having a predetermined waveform and power level are is supplied to element 134. Lumen 137 of element 134 via means within proximal handle portion 102 is in communication with tubular member 118 such that irrigant supplied by member 118 may flow to distal end 135 of element 134. Element 134 has a distal end portion 135 having a beveled distal surface 136 offset from axis 180 angle 138. Distal portion 135 is surrounded by tubular insulator 140 having a beveled distal surface 142 offset from axis 180 angle 144, angle 144 being greater than angle 138 of beveled distal surface 136 of element 134. Insulator 140 is formed from a suitable dielectric material such as alumina, zirconia, silicon-nitrate, or another suitable ceramic or high-temperature polymeric material. Proximal portion 146 of insulator 140 is overlapped by distal portion 162 of polymeric dielectric element 160, element 160 extending proximally to the proximal end of element 134. Dielectric element 160 may be formed of a heat shrinkable tubing. Metallic tubular element 128 has a distal portion 130 and extends proximally to handle portion 102 wherein element 128 is connected by means within handle portion 102 and cable 116 to an electrosurgical generator so as to provide a return path to the generator. The exterior surface of tubular element 128 is covered by dielectric element 126 except for distal portion 130 which is distal to distal end 132 of dielectric element 126. Tubular metallic element 150 is coaxially positioned about insulator 140 and has a beveled distal surface 152 parallel to surface 142 of insulator 140 and recessed from surface 142 distance 156.

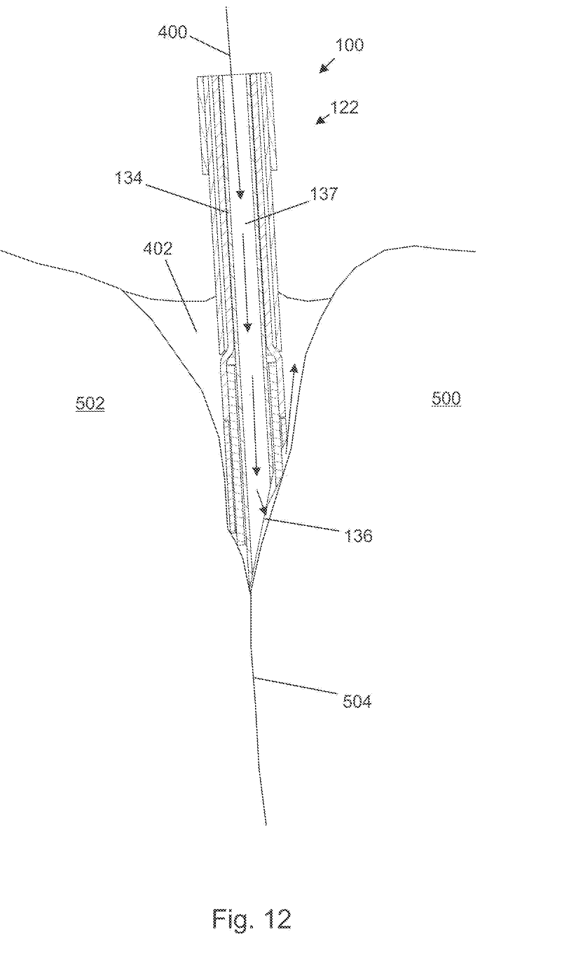

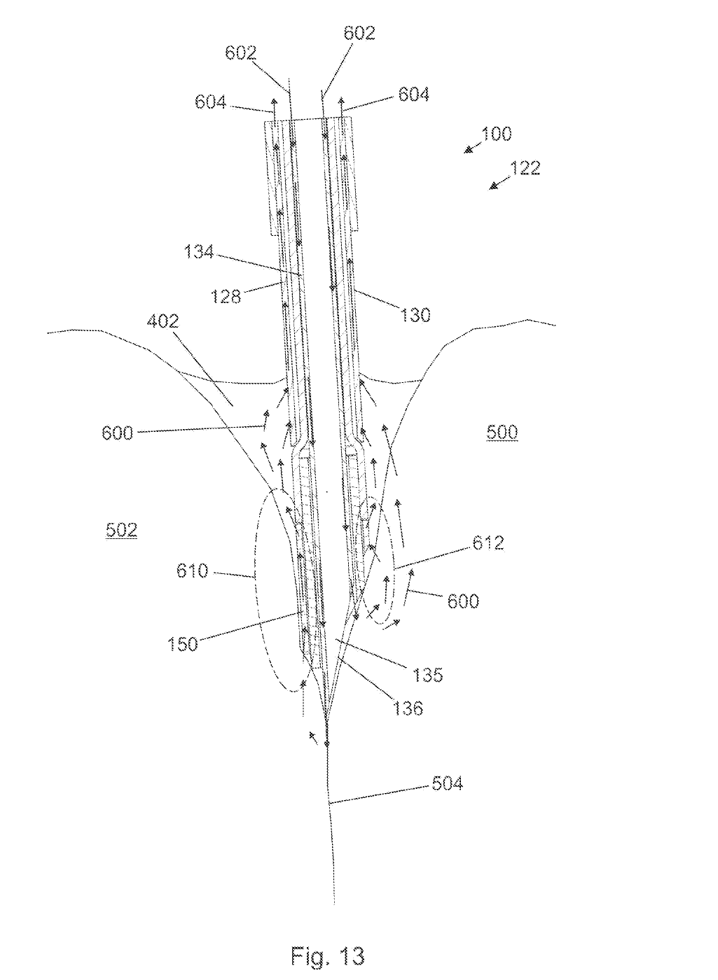

FIGS. 12 and 13 depict the distal portion of an electrosurgical device 100 formed in accordance with the principles of this invention during use excising a tissue mass. In these figures, distal portion 122 of device 100 is advanced distally while the being swept laterally in a plane perpendicular to the plane of the figure so as to separate tissue mass 500 from adjacent tissue 502 along the margin 504 between the tissues. FIG. 12 depicts the flow of irrigant 400 (indicated by arrows) through lumen 137 of tubular element 134 to the region surrounding beveled distal-most surface 136 of tubular element 134, there to collect in a localized puddle 402. Excess irrigant 400 is removed from the region by an external suction device (not shown)

FIG. 13 depicts the current flow 600 indicated by arrows. When either first button 110 or second button 112 is depressed, current 602 flows from the electrosurgical generator via cable 116 and means within handle 102 to tubular element 134, and therethrough to beveled distal end 135. The distal portion of beveled distal surface 136 forms a cutting edge. As distal portion 122 of device 100 is swept laterally in a plane perpendicular to the plane of the figure and advanced distally along the margin 504 between tissue mass 500 from adjacent tissue 502, high density RF current at the distal cutting edge of distal surface 136 resects tissue in close proximity. Current 600 flows from uninsulated portions of distal end 135 of tubular element 134 through tissue and conductive irrigant along paths indicated by arrows in FIG. 13 to portions of uninsulated region 130 of tubular element 128 which are in contact with conductive irrigant puddle 402 and which forms a return electrode, and therethrough to conductive means within proximal handle portion 102 of device 100 and cable 116 (indicated by current element 604) to the return of the electrosurgical generator. A portion of the current, indicated by regions 610 and 612, flows through tubular element 150 in the path between distal portion 135 of element 134 which functions as an active electrode and uninsulated region 130 of tubular element 128 which functions as a return electrode. Current following this path desiccates tissue in the region between portion 135 of element 134 and the distal portion of element 150 and in the region between element 150 and uninsulated region 130 of element 128. This desiccating effect reduces bleeding so as to allow more rapid completion of the procedure.

Element 150 functions as a floating electrode. The functioning of electrosurgical devices with floating electrodes is taught in detail in U.S. Pat. Nos. 7,566,333 and 7,563,261 herein incorporated by reference in their entirety. Principal effects of a floating electrode on an electrosurgical device are to concentrate RF energy in the region surrounding the active electrode, and to concurrently produce a desiccating/haemostatic effect on tissue adjacent to this region. While this is desirable in most circumstances, there may be conditions under which a floating electrode is not necessary or desirable.

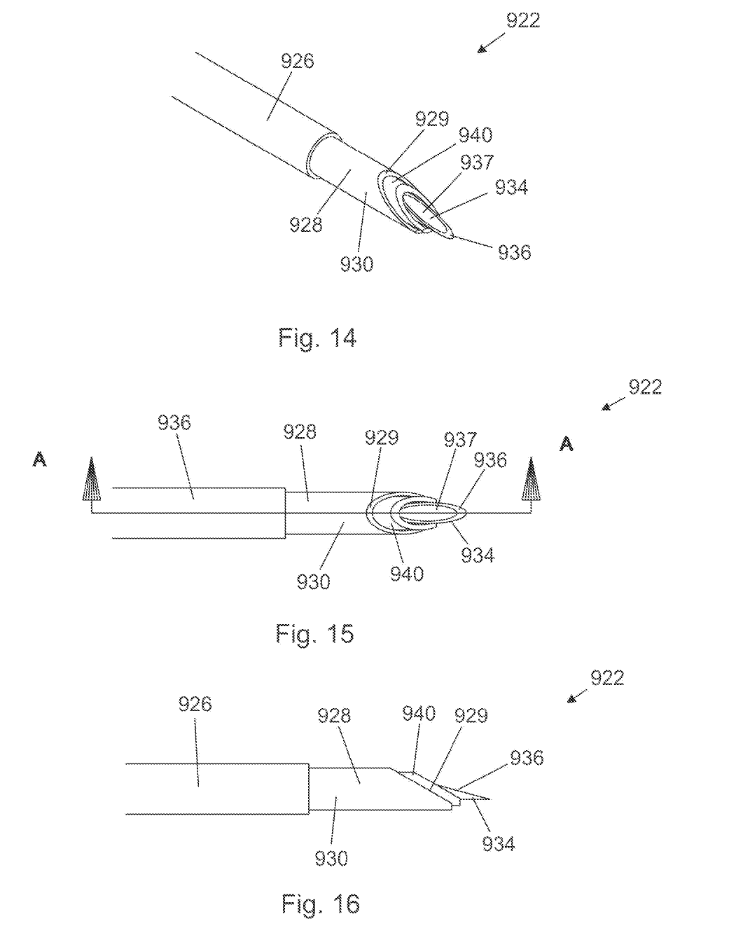

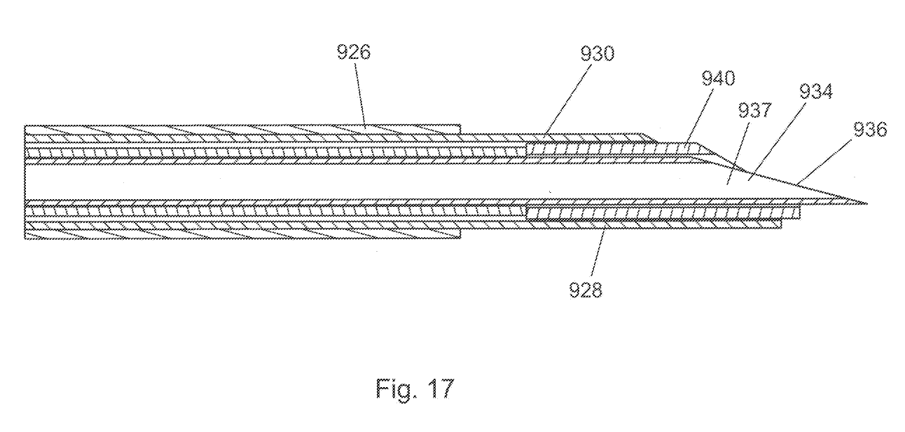

FIGS. 14 through 17 depict the distal portion (corresponding to FIGS. 8 through 11 device 100) of an alternate embodiment miniaturized electrosurgical device constructed in accordance with the principles of this invention. Device 900 differs from device 100 in that the element 500 (the floating electrode) has been eliminated. In other aspects the construction of device 900 is identical to that of device 100 except as noted. Tubular element 934 (corresponding to element 134 of device 100) has a lumen 937 and a beveled distal end with a distal-most surface 936. Insulator 940 separates distal portion 930 of tubular element 928 from tubular element 934.

Use of device 900 is identical to that of device 100 with irrigant flow and current flow identical except that the current path does not include a floating element.

While the operation of devices 100 and 900 has been described with the distal portion of the device submerged in naturally occurring body fluids and irrigant supplied from an external source through lumen 137 of element 134, it will be understood that devices 100 and 900 may also be advantageously used in environments in which external irrigant is not supplied via lumen 137 to the region of the distal tip of the devices. In such cases the site may be in a naturally occurring or created body cavity that is partially or completely filled with externally supplied irrigant that is not supplied via lumen 137 of element 134. The principles of operation of devices 100 and 900 are not affected. Indeed, when devices 100 and 900 are used in cavities completely or partially filled with externally supplied irrigant, lumen 137 of element 134 may be eliminated such that rather being tubular, element 134 is a solid rod. In such embodiments slots, ridges, or other protuberances may be added to distal surface 136 of element 134 to increase current density and increase device efficiency.

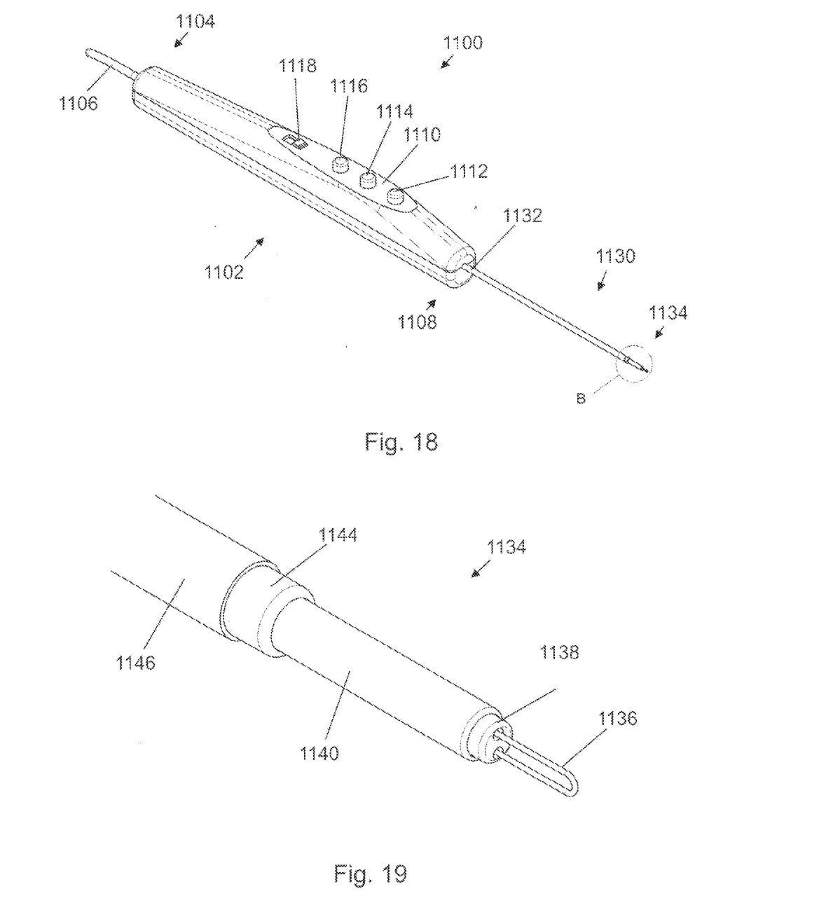

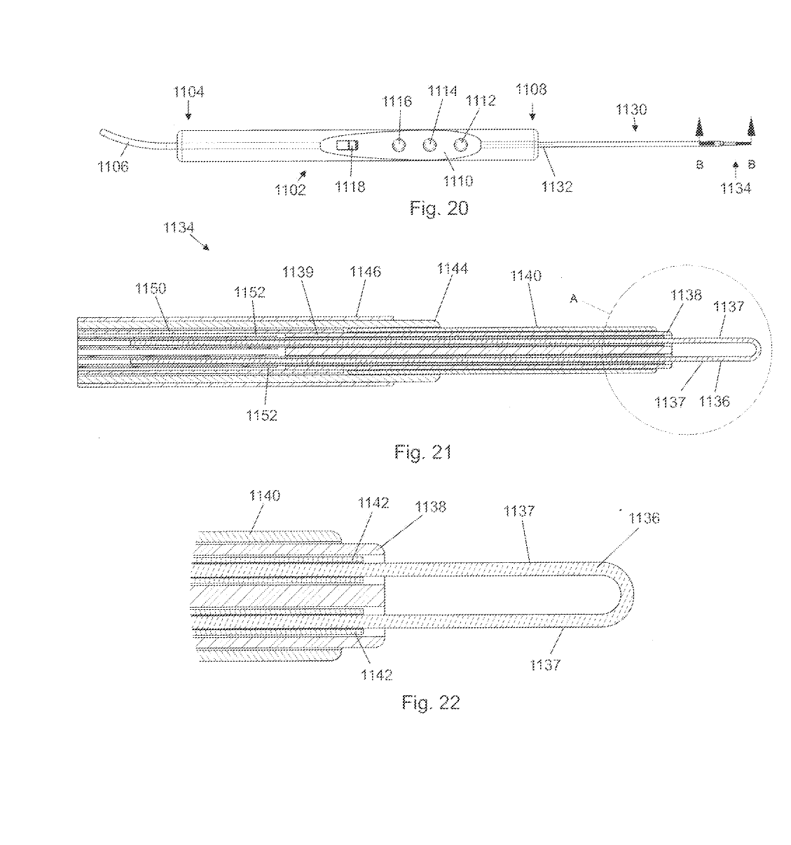

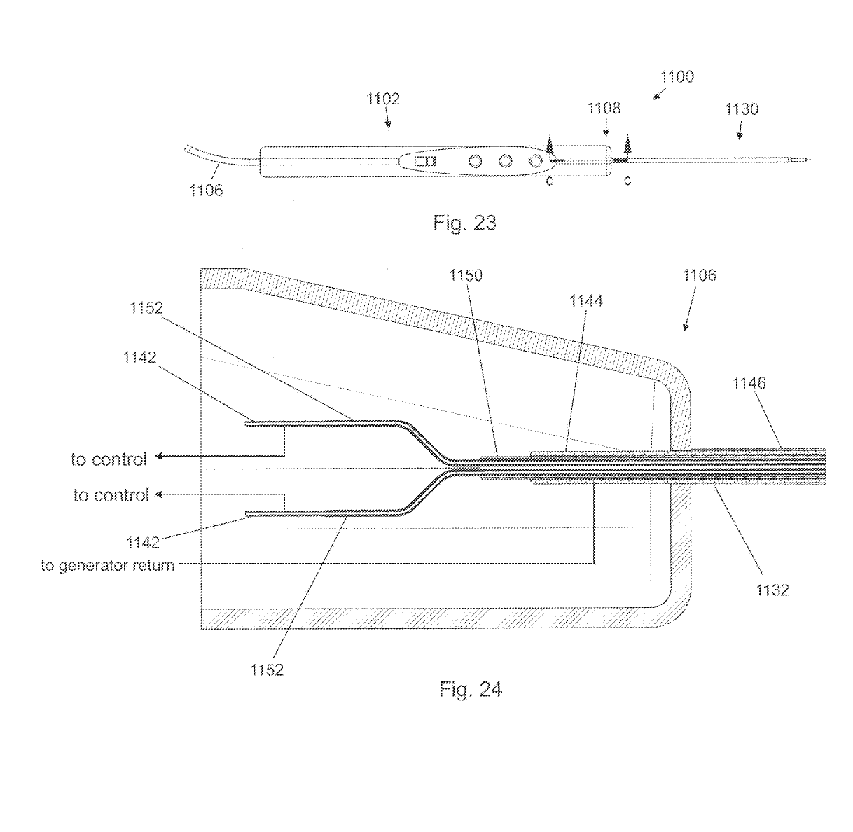

FIGS. 18 through 24 depict a dual-mode electrosurgical/thermal device constructed in accordance with the principles of this invention. In a preferred embodiment, the mode is selected by the user, via a switch on the handle of the device. The active electrode of the device is an elongate wire loop of small diameter, preferably less than 0.03 inches (0.75 mm) in diameter, and more preferably less than 0.016 inches (0.4 mm) in diameter. In a preferred embodiment, the elongate loop electrode is made of tungsten. In other embodiments, other materials are used, for instance, stainless steel, nickel, titanium or other alloys. The two proximal ends of the elongate wire loop are electrically connected to switching and control circuitry such that in a first selectable electrosurgical mode of operation, RF energy from an external electrosurgical generator is supplied to the active electrode. In a preferred embodiment, a return electrode in close proximity to the active electrode is connected via cabling to the return of the electrosurgical generator. In a preferred embodiment, the device is hand controlled when in this first electrosurgical mode such that the user may select first or second waveforms at preselected power levels for supplying to the active electrode by activating the generator through either a first or a second button on the handle. In other embodiments, the selection and activation of these waveforms (commonly referred to as “Cut” and “Coag”) is accomplished through a foot pedal. In this first electrosurgical mode in which the device has a return electrode in close proximity to the active electrode (a bipolar configuration), current flows from the active electrode through tissue and conductive fluid with which it is in contact to portions of the return electrode which are also in contact with the tissue and/or conductive fluids. For alternate embodiments in which the return electrode is mounted on the patient in a remote location using a standard dispersive pad (i.e., a monopolar configuration), current flows from the active electrode through tissue and conductive fluid with which it is in contact to the remotely located return electrode, and therethrough via cabling to the generator return. For applications such as, for instance, brain surgery in which stray electrical currents may cause damage to surrounding tissue use of a bipolar configuration is preferable, while in others the monopolar configuration is preferred. When operating in a dry environment, the monopolar configuration is indicated since the lack of conductive fluid may interrupt the return path via the return electrode on the device when using the bipolar configuration. In a selectable second thermal mode, energy is supplied to the elongate loop electrode such that current flows from the switching and control circuitry to a first end of the elongate loop electrode through the wire of the electrode and through the second end back to the switching and control circuitry. Current flowing through the loop electrode heats the electrode so that thermal energy rather than RF energy may be applied to tissue. Since no current is flowing from the electrode into the tissue, there is no chance of damage to adjacent tissue due to current flow and no interference with items such as pacemakers, other implanted devices or sensing/stimulating electrodes attached to the patient. Activation is through a button control on the handle of the device. In certain embodiments, current flows through the elongate loop electrode during the entire period that the button is depressed. In other embodiments, each activation causes current flow for a predetermined period of time or a predetermined energy. In certain embodiments, a potentiometer, or an electronic regulating circuit in the current path allows the user to adjust the degree of heating of the elongate electrode when in the thermal mode. In a preferred embodiment, when operating in the second thermal mode, current flow to the elongate loop electrode from the switching and control circuitry is supplied to that circuitry by a battery pack inside of the handle of the device, the energy from the battery being direct current. In other embodiments, the energy is supplied to the switching and control circuitry by the electrosurgical generator either as RF energy or as low frequency or DC. The energy supplied to the elongate loop electrode when in thermal mode can be RF, rectified RF, low frequency alternating current or direct current.

FIGS. 18 through 24 depict a dual-mode electrosurgical/thermal device 1100 constructed in accordance with the principles of this invention. Device 1100 has a proximal handle portion 1102 with a proximal end 1104 from which passes cable 1106 which is connected to an electrosurgical generator (not shown). Handle portion 1102 has a distal end 1106 from which protrudes elongate distal portion 1130, and a top surface 1110 in which are positioned first, second and third activation buttons 1112, 1114 and 1116 respectively along with slide switch 1118. Elongate distal portion 1130 has a proximal end 1132 mounted to distal end 1108 of handle portion 1102, and a distal assembly 1134. As best seen in FIGS. 19, 21 and 22, distal assembly 1134 has a insulator 1138 formed of alumina or another suitable high-temperature dielectric material, the insulator having two lumens from which protrude the elongate axial portions 1137 of metallic loop electrode 1136, electrode 1136 being preferably formed of a small-diameter wire. In a preferred embodiment tungsten wire is used. In other embodiments, other materials are used, for instance, stainless steel, nickel, titanium or other alloys. The diameter of the wire used to form electrode 1136 is preferably less than 0.03 inches (0.75 mm) in diameter, and more preferably less than 0.016 inches (0.4 mm) in diameter. As best seen in FIG. 22, the proximal portions of elongate axial portions 1137 of electrode 1136 are affixed to elongate metallic tubular members 1142 by welding, soldering, crimping or some other means, tubular members 1142 extending proximally to the interior of handle portion 1102 so as to provide a pathway for electrical current between circuitry within handle 1102 and electrode 1136. Tubular dielectric members 1152 insulate the outer surfaces of tubular members 1142 and extend from the proximal end of insulator 1138 to the interior of handle portion 1102. Metallic tubular member 1140 is coaxially positioned about the exterior of insulator 1138 and extends proximally so that proximal end 1139 of insulator 1138 protrudes beyond the proximal end of tubular member 1140. Tubular dielectric member 1150 overlaps the proximal end 1139 of insulator 1138 and extends proximally to the interior of interior of handle portion 1102. Metallic tubular member 1140 protrudes distally from the distal end of metallic member 1144 to which it is affixed by welding, brazing or mechanical press fit, member 1144 extending proximally to the interior of handle portion 1102. Dielectric tubular member 1146 is coaxially positioned about metallic tubular member 1144 from near the distal end of metallic tubular member 1144 to distal end 1108 of handle portion 1102. Referring to FIG. 24 depicting in section view distal end 1108 of handle portion 1102 and proximal portion 1132 of elongate distal portion 1130, metallic tubular members 1142 are insulated from each other by dielectric tubular members 1152. Dielectric tubular member 1150 provides additional insulation between tubular members 1142 and metallic tubular member 1144.

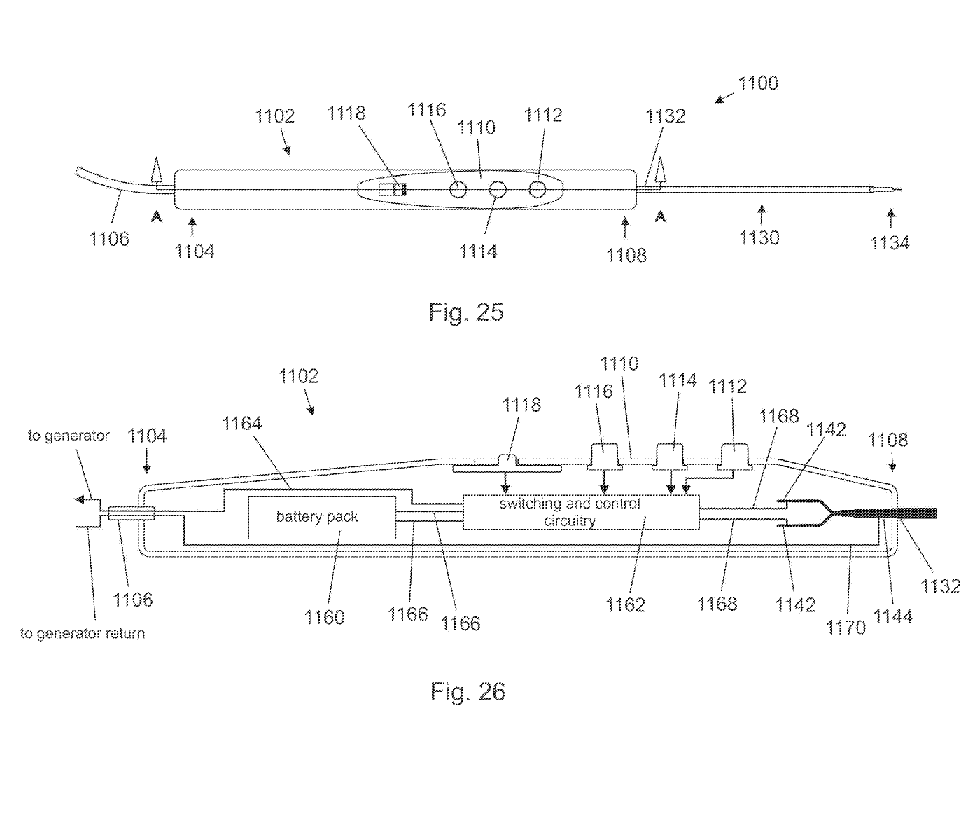

FIGS. 25 and 26 diagrammatically depict handle portion 1102 together with its internal components of a preferred embodiment of a dual mode electrosurgical/thermal device constructed in accordance with the principles of this invention. Switching and control circuitry module 1162 is connected via cable 1106 and internal circuitry 1164 to an electrosurgical generator (not shown), and via circuitry 1166 to battery pack 1160. switching and control circuitry module 1162 is also connected via circuitry 1168 to elongate tubular members 1142 and therethrough to elongate axial portions 1137 of loop electrode 1136 so as to provide current paths between switching and control circuitry module 1162 and loop electrode 1136. The proximal portion of metallic tubular member 1144 is connected via circuitry 1170 and cable 1106 to the return of the electrosurgical generator so as to provide a current path from the return of the electrosurgical generator to metallic tubular element 1140. Slide switch 1118 together with the switching and control circuitry module determines the mode of operation of the device.

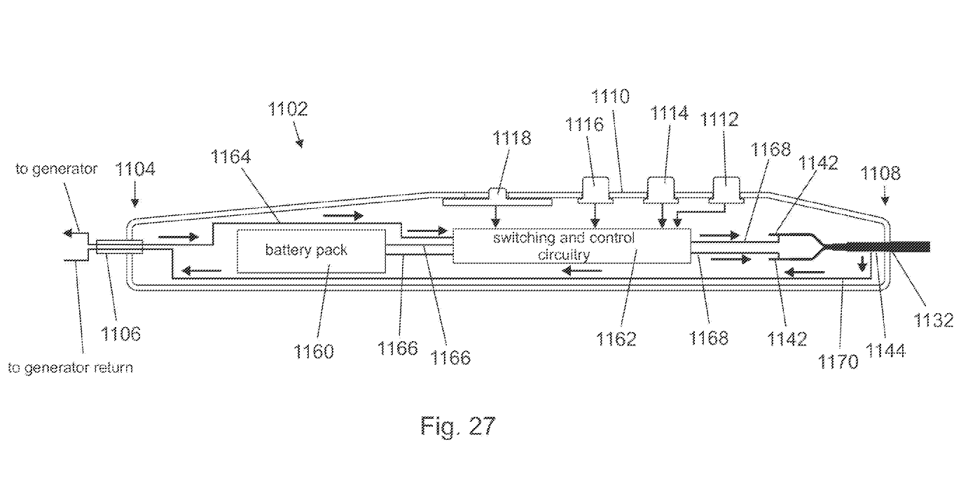

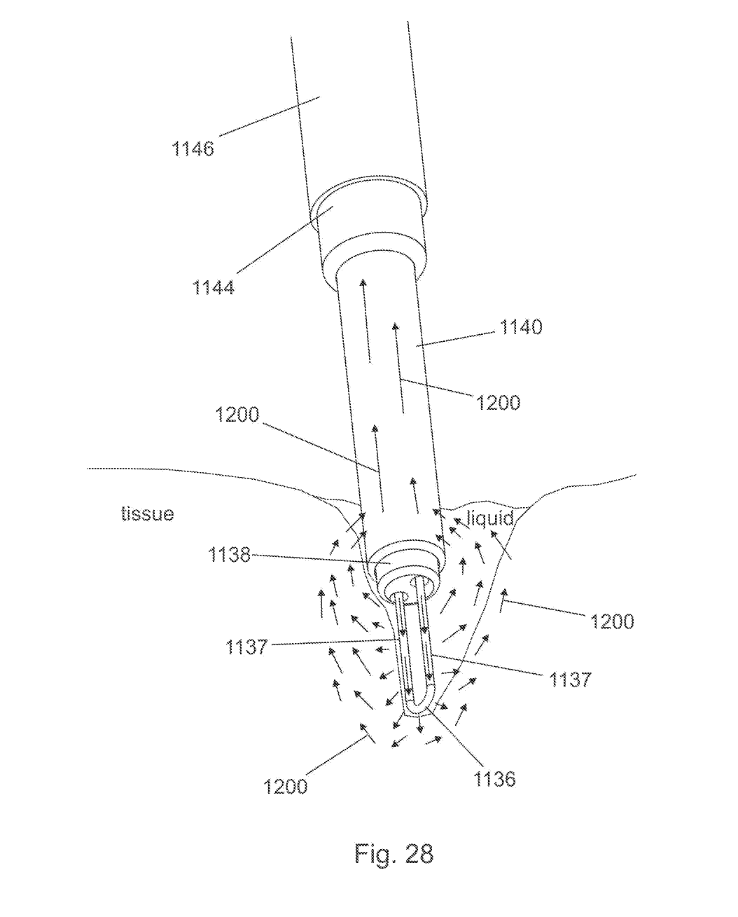

Referring now to FIGS. 27 and 28, a preferred embodiment of device 1100 is depicted having two modes of operation: either as an electrosurgical device having a return electrode in proximity to the active electrode, or as a thermal device. Slide switch 1118 is in a first position in which first button 1112 and second button 1114 control the output of the electrosurgical generator such that depressing first button 1112 causes RF energy having a first waveform and power level to active electrode 1136, and depressing second button 1114 causes RF energy having a second waveform and power level to active electrode 1136. Radio Frequency energy supplied by the generator, via cable 1160 and circuitry 1164 to switching and control circuitry module 1162, is conducted via circuitry 1168 and elongate conductive tubular members 1142 to axial portions 1137 of electrode 1136. A return path to the generator is provided by tubular metallic element 1140, elongate metallic tubular element 1144, circuitry 1170 and cable 1106 which is electrically connected to the return receptacle of the generator. FIG. 28 depicts the distal end of device 1100 while electrosurgically cutting tissue, conductive liquid surrounding active electrode 1136 and the distal portion of tubular metallic element 1140 which functions as a return electrode. Arrows 1200 depict the current flow in vicinity to active electrode 1136 and return electrode 1140. Tissue contacting active electrode 1136 in the direction of travel 1202 is vaporized. Because active electrode 1136 and return electrode 1140 are in close proximity current flow is largely confined to the region surrounding the two electrodes. Current flow through tissue in the return current path between active electrode 1136 and return electrode 1140 is desiccated by the current thereby providing control of bleeding due to the current's desiccating coagulating effect. The conductive liquid may be body fluids such as blood or an externally applied irrigant. Conductive irrigant such as saline may be used. When a non-conductive irrigant such as sterile water or glycine is used, contamination by body fluids and byproducts of the cutting process impart to the irrigant sufficient conductivity for device 1100 to function in the manner described.

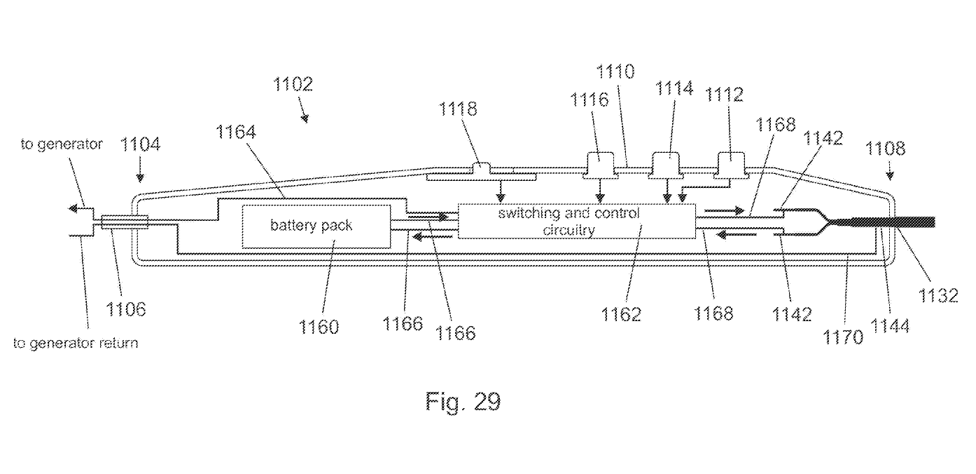

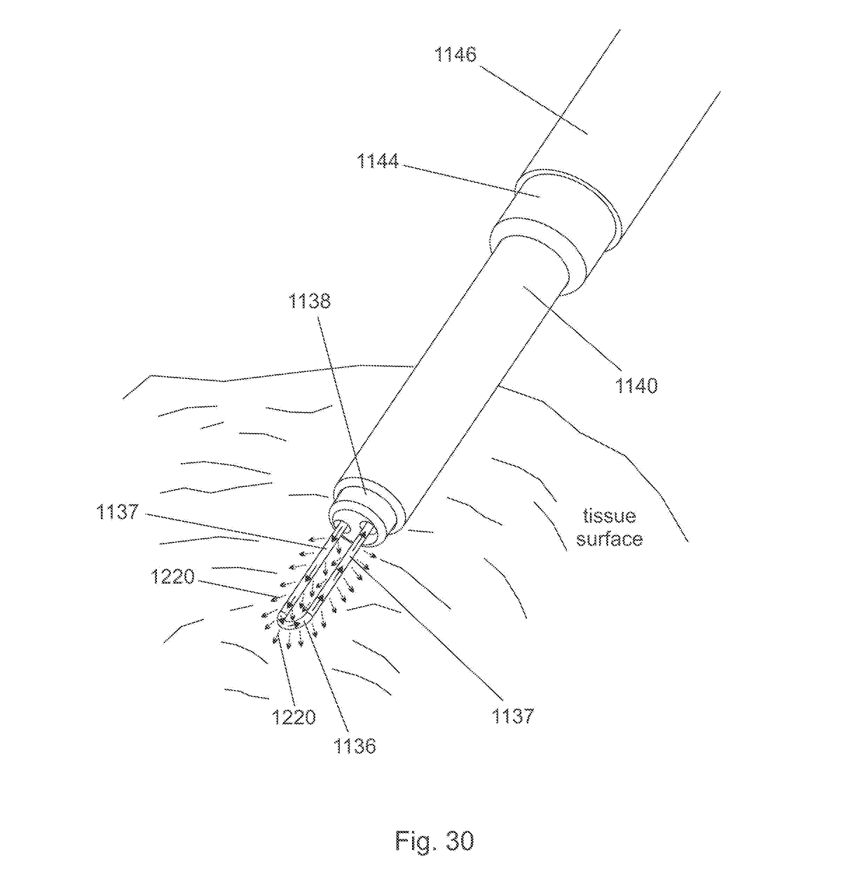

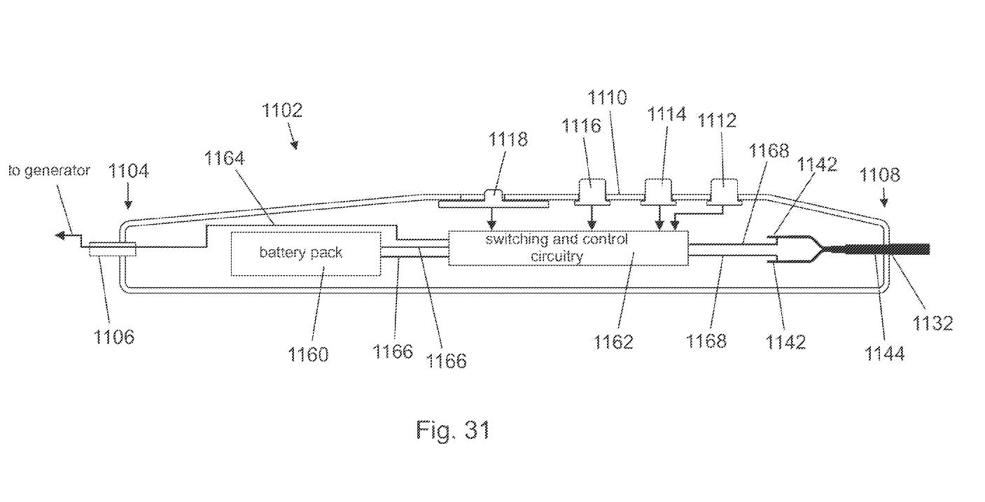

FIG. 29 diagrammatically depicts device 1100 in its second thermal mode, switch 1118 being in its second position. When device 1100 is activated by third button 1116, current flows from battery pack 1160 via a first 1116 conductive path to switching and control circuitry module 1162, and therethrough via first circuitry 1142 and a first elongate conductive tubular element 1140 to a first axial portion 1137 of loop electrode 1136. Current flows through loop electrode 1136, through second axial portion 1137, through second elongate conductive tubular element 1140 to a second circuitry 1142 to switching and control circuitry 1162 and finally to battery pack 1160. Current flowing through loop electrode 1136 causes resistive heating of electrode 1136. As best seen in FIG. 30 depicting the distal portion of device 1100, thermally treating a tissue surface, current flow is through electrode 1136 only. There is no current flow from electrode 1136 to the patient. Thermal energy only (represented by dashed arrows 1210) is used to affect tissue when device 1100 is used in its second thermal mode. In certain embodiments the current flow through electrode 1136 and its resulting temperature may be adjusted by a potentiometer in the current path or by circuitry within the switching and control circuitry module 1162.

In alternate embodiments, of device 1100 the battery pack is eliminated and the energy source for the thermal mode is remotely located. In a preferred embodiment, an electrosurgical generator is the energy source. In certain embodiments, the electrosurgical generator is a special purpose unit having a socket for providing direct current power or rectified RF energy to device 1100 for the thermal mode of operation. In other embodiments, a general purpose generator is used, the switching and control circuitry module 1162 further including circuitry for converting an RF signal from the generator to a voltage and frequency suitable for the thermal function of device 1100. In a preferred embodiment, device 1100 has only first button 1112 and second button 1114, first button 1112 being used for supplying RF energy to electrode 1137 in the first electrosurgery mode, and second button 1114 causing the electrosurgical generator to supply to switching and control circuitry 1162 RF energy having a power level and waveform suitable for rectifying and supplying to electrode 1136 when device 1100 us used in the second thermal mode.

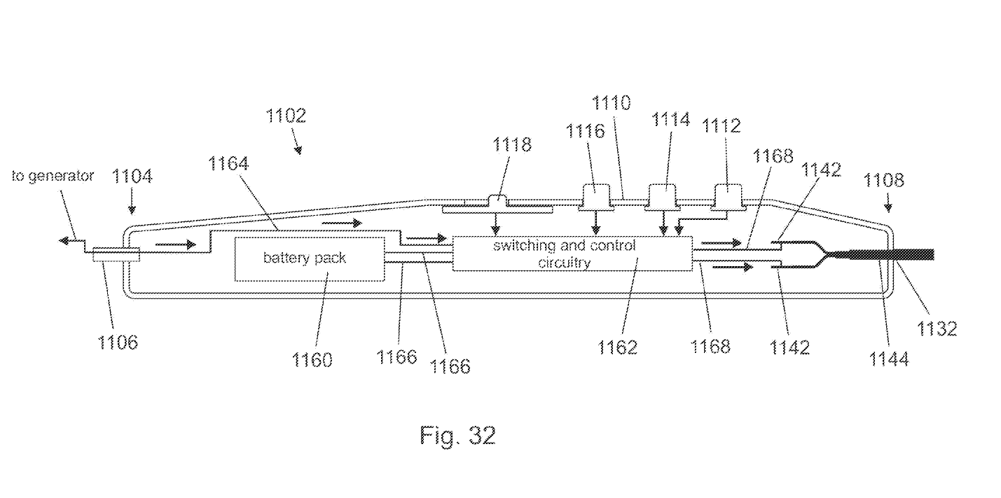

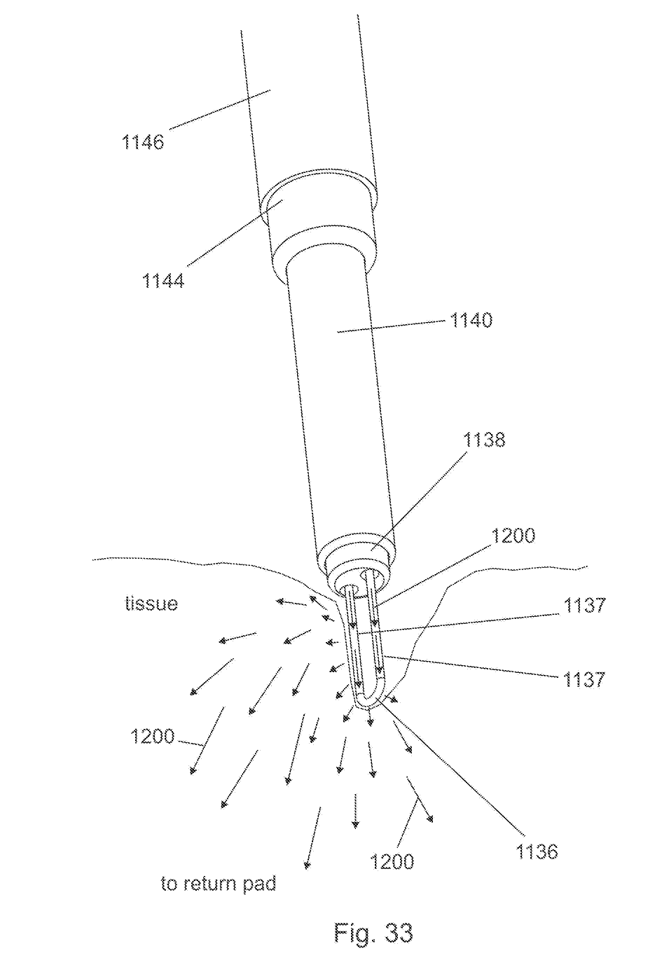

While device 1100 as previously herein described may be used in surgical procedures in which some liquid is present to complete the current path back to the generator via return electrode 1140 and its associated circuitry, it cannot be used in dry environments. Accordingly, there is a need for an electrosurgical device having electrosurgical and thermal modes which can be used in dry environments. FIG. 31 diagrammatically depicts the handle portion 1102 of a monopolar embodiment of device 1300, that is, an embodiment in which the device does not have a return electrode in proximity to active electrode 1136, but rather is used with a remotely located dispersive electrode (return pad). In all regards device handle portion 1102 as depicted in FIG. 31 is identical to handle portion 1102 of FIG. 26 except that, referring to FIG. 26, conductive path 1170 to metallic tubular element 1144 has been eliminated. Referring to FIGS. 32 and 33 which diagrammatically depicts the handle portion 1102 of monopolar embodiment 1300 when in use in the first electrosurgical mode, switch 1118 being in its first position. In all aspects the operation of handle portion 1102 of monopolar embodiment 1300 as depicted in FIG. 32 is identical to that of bipolar embodiment 1100 as depicted in FIG. 27 except that the return path to the electrosurgical generator is not through a return electrode on the device but rather through a remotely located return electrode. FIG. 33 depicts distal portion 1134 of elongate distal portion 1130 of monopolar embodiment 1300 during use electrosurgically cutting tissue. Because conductive fluid is not present, current 1200 flows only from portions of active electrode 1136 which are in contact with tissue. Current flows through the tissue to the remotely located return pad and therethrough to the return receptacle of the electrosurgical generator. While the monopolar embodiment 1300 is able to cut tissue in dry environments, the desiccating effect created by bipolar embodiment 1100 working in a wet environment is absent since there is no current flow to tubular element 1140 which functions as the return electrode in bipolar embodiment 1100.

Device 1100 is a bipolar electrosurgical device configured for the cutting of tissue in a wet or semi-dry environment wherein the active and return electrodes are configured to allow the cutting of tissue at extremely low power levels and with the electric field intensity highly concentrated at the device distal tip. Device 1100 may be advantageously used in procedures such as, for instance, brain surgery in which stray electrical currents may cause damage to surrounding tissue.

### INDUSTRIAL APPLICABILITY

The dual-mode electrosurgical devices and methods of the present invention allow a single device to both cut, resect, or otherwise ablate a target tissue of interest and also thermally cauterize (or “spot coagulate”) bleeding tissues to achieve full haemostasis before removal. The use of a singly introduced device where previously multiple incisions, insertions, and/or instruments were required not only desirably simplifies the overall procedure but also minimizes patient trauma and thus speeds recovery time. In addition, the bipolar construction eliminates the risk of complications arising from uncontrolled or non-uniform current dispersal to tissues other than those targeted. Accordingly, the present invention fills a lingering need not served by current electrosurgical alternatives.

All patents and publications mentioned herein are incorporated by reference in their entirety. Nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Such other advantages and features will become apparent from the claims filed hereafter, with the scope of such claims to be determined by their reasonable equivalents, as would be understood by those skilled in the art. Thus, the invention is defined not by the above description, but by the following claims and their equivalents.

What is claimed:

1. A dual-mode electrosurgical/thermal device comprising:
a. a proximal handle portion having a proximal end configured for connection to an external power source, wherein said handle portion contains a switching and control circuitry module that allows the device to be operated in either a first electrosurgical mode or a second thermal mode;
b. an elongate tubular portion affixed to the distal end of said handle portion;
c. a distal assembly affixed to the distal end of said elongate tubular portion that comprises:
i. an insulator; and
ii. an active element formed from an electrically conductive material that is in electrical communication with said external power source, wherein said active element takes the form of an elongate wire loop comprising of first and second axial sections and a third tranverse section, wherein the respective proximal ends of said first and second axial sections are affixed to the distal end of said insulator and the respective distal ends of said first and second axial sections connected to each other by means of the third transverse section;
d. circuity and cabling that extends from the proximal end of the handle portion to said distal assembly so as to define a current path from handle portion to said active element and back;
wherein said active element operates as a current-dispersing ablation electrode when the device is in the first electrosurgical mode and as a cauterizing heating element when said device is in the second thermal mode.

2. The dual-mode electrosurgical/thermal device of claim 1, wherein said device is a monopolar device that operates in conjunction with a return electrode in form of a remotely mounted dispersive pad.

3. The dual-mode electrosurgical/thermal device of claim 1, wherein said distal assembly further comprises a return electrode in close proximity to the active element.

4. The dual-mode electrosurgical/thermal device of claim 1, wherein said distal assembly further comprises a conductive floating electrode that is not in electrical communication with any power source positioned in close proximity to the active element, wherein the floating electrode coordinates with the active element to concentrate the power in the vicinity of the active element and increase the energy density in the region surrounding the active element.

5. The dual-mode electrosurgical/thermal device of claim 1, wherein when the device is operating in the electrosurgical mode, the energy supplied to the active element is RF energy.

6. The dual-mode electrosurgical/thermal device of claim 1, wherein when the device is operating in the thermal mode, the energy supplied to the active element is selected from the group consisting of rectified RF energy, low frequency alternating current or direct current.

7. The dual-mode electrosurgical/thermal device of claim 1, wherein said external source comprises an electrosurgical generator capable of supplying a radio-frequency (RF) signal.

8. The dual-mode electrosurgical/thermal device of claim 1, wherein said switching and control circuitry module further includes circuitry for converting an RF signal from an external power source to a voltage and frequency suitable for operating in said active element in said second thermal mode.

9. The dual-mode electrosurgical/thermal device of claim 1, wherein said switching and control circuitry module further includes a potentiometer or an electronic regulating circuit in the current path that allows the user to adjust the degree of heating of the active element when the device is operating in the thermal mode.

10. The dual-mode electrosurgical/thermal device of claim 1, wherein said handle portion further comprises an onboard power source.

11. The dual-mode electrosurgical/thermal device of claim 1, wherein said onboard power source comprises a battery pack.

12. The dual-mode electrosurgical/thermal device of claim 1, wherein said handle portion comprises an array of activation and control elements that coordinate with said switching and control circuitry module to direct the operation of the device in said first electrosurgical mode or said second thermal mode.

13. The dual-mode electrosurgical/thermal device of claim 12, wherein said an array of activation and control elements includes:
a. a first button for activating the application of an RF signal having a first waveform and preselected power level from the external power source to both said first and second axial sections of the active element;
b. an optional second button for activating the application of an RF signal having a second waveform and preselected power level from the external power source to both said first and second axial sections of the active element;
c. a third button for activating the application of electrical current to only the first axial section of said active element, whereby current flows from the first to the third to the second section of said active element so as to cause resistive heating of the element.

14. The dual-mode electrosurgical/thermal device of claim 13, wherein said an array of activation and control elements further includes a slide switch for alternating between electrosurgical mode and thermal mode.

15. The dual-mode electrosurgical/thermal device of claim 14, wherein said slide switch alternates the power source from an external power source of RF energy to an on-board power source of direct current.

16. The dual-mode electrosurgical/thermal device of claim 1, wherein said device is miniaturized to facilitate atraumatic introduction into the tissues of the brain via an incision in the skull.

17. The dual-mode electrosurgical/thermal device of claim 3, wherein the elongate wire loop and the return electrode are each formed from an electrically conductive material selected from the group consisting of tungsten, stainless steel, nickel, titanium and alloys thereof.

18. The dual-mode electrosurgical/thermal device of claim 1, wherein the elongate wire loop has a small diameter on the order of less than 0.03 inches (0.75 mm).

19. The dual-mode electrosurgical/thermal device of claim 1, wherein the elongate wire loop has a small diameter on the order of less than 0.016 inches (0.4 mm).

20. The dual-mode electrosurgical/thermal device of claim 1, wherein said insulator is formed of alumina or another suitable high-temperature dielectric material.

21. A method for removing a brain tumor in a patient in need thereof, said method comprising the steps of:
a. Introducing the dual-mode electrosurgical/thermal device of claim 1 through an incision in the patient's skull and advancing the device to be in contact with the brain tumor;
b. Selecting the electrosurgical mode such that said active element operates as a current-dispersing ablation electrode and manipulating said active element so as to ablate or resect the brain tumor; and
c. Selecting the thermal mode such that said active element operates as a cauterizing heating element and manipulating said active element to spot coagulate bleeding tissues.

22. The method of claim 21, wherein said device is miniaturized to facilitate atraumatic introduction into the tissues of the brain via an incision in the skull.

23. The method of claim 21, wherein the distal assembly of said dual-mode electrosurgical/thermal device further comprises a return electrode positioned in close proximity to the active element.

24. The method of claim 23, wherein the distal assembly of said dual-mode electrosurgical/thermal device further comprises a conductive floating electrode that is not in electrical communication with any power source positioned in close proximity to the active element, wherein the floating electrode coordinates with the active element to concentrate the power in the vicinity of the active element and increase the energy density in the region surrounding the active element.

25. The method of claim 21, wherein said selection step (b) involves pressing a first button that activates the application of an RF signal having a first waveform and preselected power level from the external power source to both said first and second axial sections of the active element and said selection step (c) involves pressing a third button that activates the application of electrical current to only the first axial section of said active element, whereby current flows from the first to the third to the second section of said active element so as to cause resistive heating of the element.

26. The method of claim 25, wherein current flows to or through the active element during entire period that the button is depressed.

27. The method of claim 25, wherein each activation causes current flow for a predetermined period of time or a predetermined energy.
